(19) United States
(12) Patent Application Publication
(10) Pub. No.: US 2006/0195115 A1
(43) Pub. Date: Aug. 31, 2006

(54) Method and apparatus for kyphoplasty

(76) Inventor: Bret A. Ferree, Cincinnati, OH (US)

Correspondence Address: JERROLD J. LITZINGER, 2134 MADISON ROAD, CINCINNATI, OH, 45208, US

(21) Appl. No.: 11/360,867

(22) Filed: Feb. 23, 2006

Related U.S. Application Data
(60) Provisional application No. 60/655,372, filed on Feb. 23, 2005.

(51) Int. Cl. A61F 2/34

(52) U.S. Cl. 606/92

(57) ABSTRACT

A method for performing kyphoplasty is disclosed. A catheter having a balloon-like component is located at its distal end, is inserted into a fractured vertebra. A substance such as PMMA to fill the balloon and stabilize the fracture is injected through the catheter. The balloon is held in place until the material begins to set.

### CROSS REFERENCE TO RELATED APPLICATIONS

This application claims benefit from U.S. Provisional Application Ser. No. 60/655,372, filed Feb. 23, 2005, which application is incorporated herein by reference.

### BACKGROUND OF THE INVENTION

1. Field of the Invention

The present invention relates generally to kyphoplasty, and, in particular, to a novel method and apparatus for performing kyphoplasty.

2. Description of the Prior Art

Over one hundred thousand people suffer compression fractures of the spine each year. The number of people with compression fractures is expected to increase, as the population ages. Compression fractures are generally caused by osteoporosis. Weak osteoporotic vertebrae may be fractured with minimal trauma. Compression fractures cause pain and spinal deformity.

Compression fractures may be created with activity modification, pain medication, medications to increase bone density, injection of Polymethylmethacrylate (PMMA), or surgical correction with rods and screws.

Injection of PMMA has become increasingly popular. Patients often experience immediate pain relief following injection of PMMA. The PMMA may be injected directly into the fractured vertebra. Alternatively, the PMMA may be injected into a cavity created in the fractured vertebra, a procedure known as kyphoplasty. Kyphoplasty elevates the fractured vertebral fragment or fragments. PMMA is placed under the elevated fragments to hold the fragments in the proper alignment. Kyphoplasty restores the proper size of the vertebra and thus, the proper alignment of the spine.

### SUMMARY OF THE INVENTION

It is therefore an object of the present invention to provide a method for kyphoplasty having fewer steps than prior art techniques.

It is a further object of the present invention to provide a method in which a portion of the PMMA used is contained within the device.

It is still a further object of the present invention to provide a method in which the PMMA used is not at risk of passing into the patient's vascular system or the spinal canal.

It is also an object of the present invention to provide a device which is constructed to cause more expansion than radial expansion.

The device may be used in any bone or other tissue within the body. For example, the invention may be used to treat fractures of the radius.

### BRIEF DESCRIPTION OF THE DRAWINGS

FIG. 1A is a lateral view of a prior art kyphoplasty device.

FIG. 1B is a lateral view of the prior art device drawn in FIG. 1A.

FIG. 1C is a sagittal cross section of a fractured vertebra and a lateral view of the device drawn in FIG. 1A.

FIG. 1D is a sagittal cross section of a fractured vertebra and a lateral view of the device drawn in FIG. 1B.

FIG. 1E is a sagittal cross section of the fractured vertebra and a lateral view of the balloon catheter drawn in FIG. 1A.

FIG. 2A is a lateral view of the preferred embodiment of the invention.

FIG. 2B is a cross section of the embodiment of the invention drawn in FIG. 2A.

FIG. 2C is a lateral view of the invention drawn in FIG. 2A.

FIG. 2D is a lateral view of the invention drawn in FIG. 2C.

FIG. 3A is a lateral view of a fractured vertebra and the embodiment of the invention drawn in FIG. 2A.

FIG. 3B is a sagittal cross section of a fractured vertebra and the embodiment of the invention drawn in FIG. 3A.

### BRIEF DESCRIPTION OF A PREFERRED EMBODIMENT

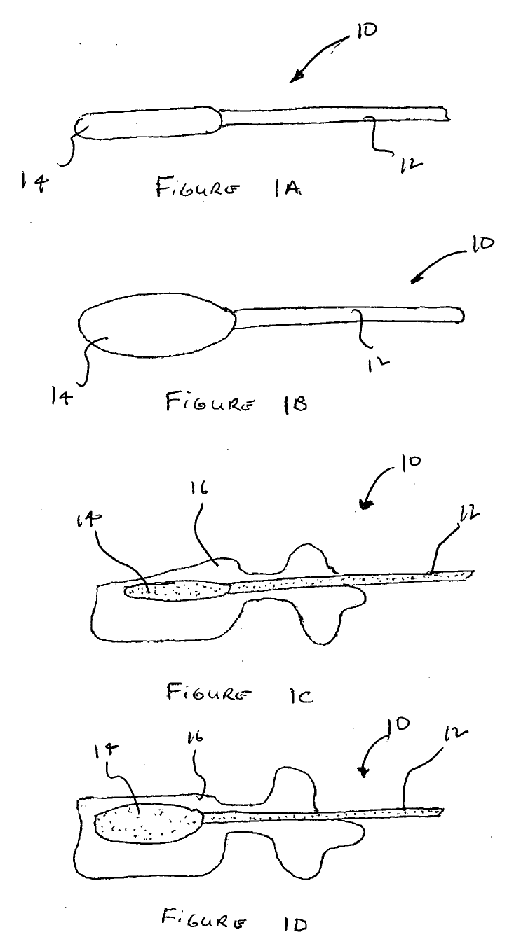

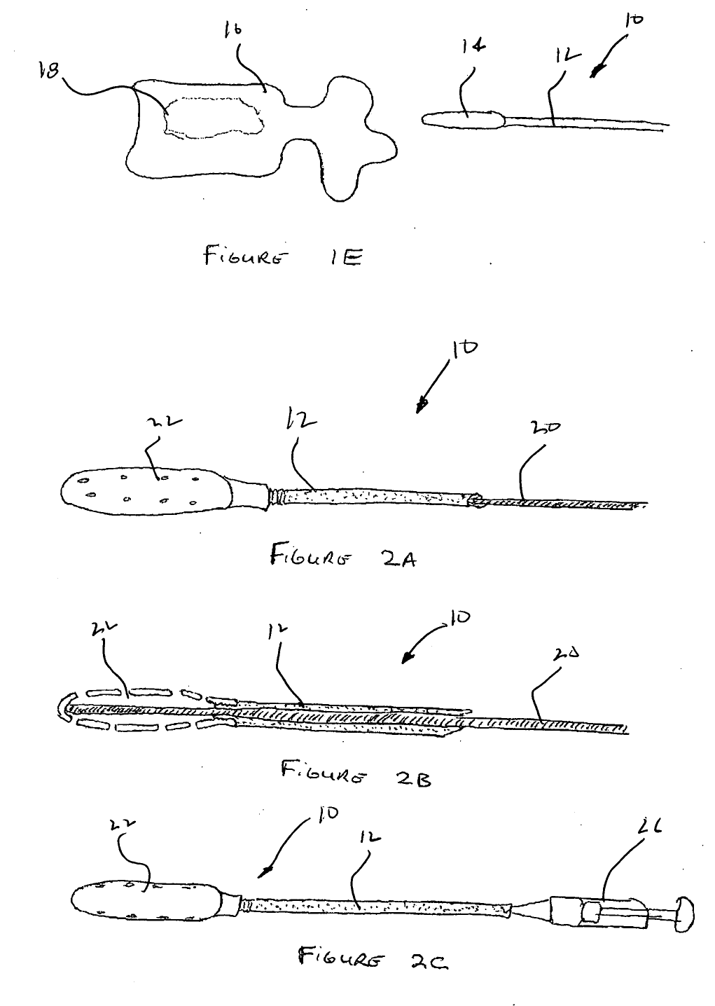

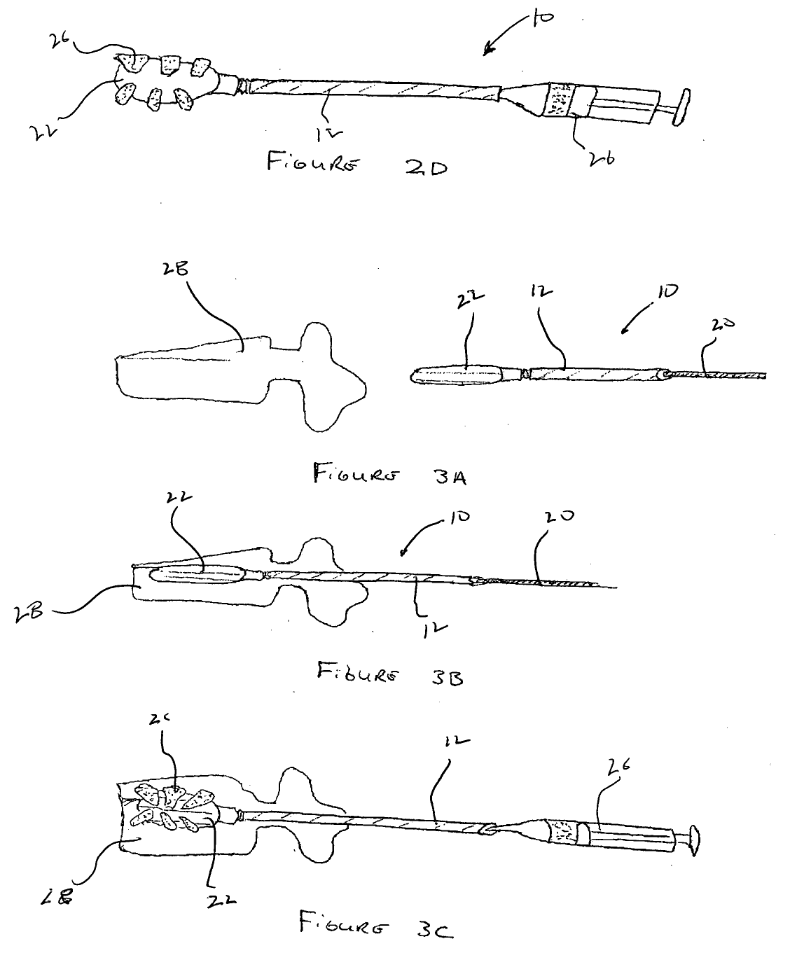

Referring now to FIG. 1A, there is shown a device for use in performing kyphoplasty on the spine of a human. The device, generally indicated at 10, is made up of a catheter 12 having a balloon 14 attached at one end. FIG. B is a depiction of device 20 shown with balloon 14 inflated, by pumping air through catheter 12 into balloon 14.

FIG. 1C shows a device 10 positioned within a fractured vertebrae 16 in its collapsed state. Balloon 14 is inserted between the vertebra adjacent the fractured site. When the device 10 is located in the proper position, as shown in FIG. 1D, the balloon 14 is inflated to expand the fractured vertebra 16. FIG. 1E shows device 10 removed from the vertebra 16. The cavity 18 created in the vertebra 16 by the balloon catheter will be filled with in-situ curing PMMA. Bioactive “cements” such as calcium phosphate, hydroxyapatite, carbonated apatite cement, and glass-ceramic powders could be used rather than PMMA. Other bio-compatible in-situ curing materials may be used such as polyurethane, hydrogel, or bioactive glues.

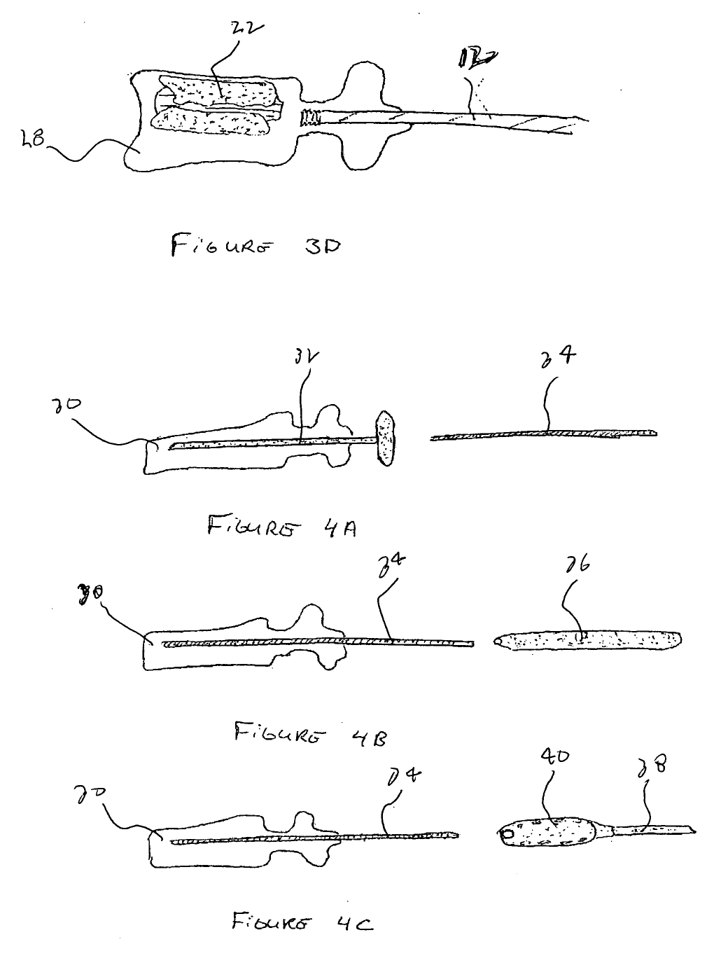

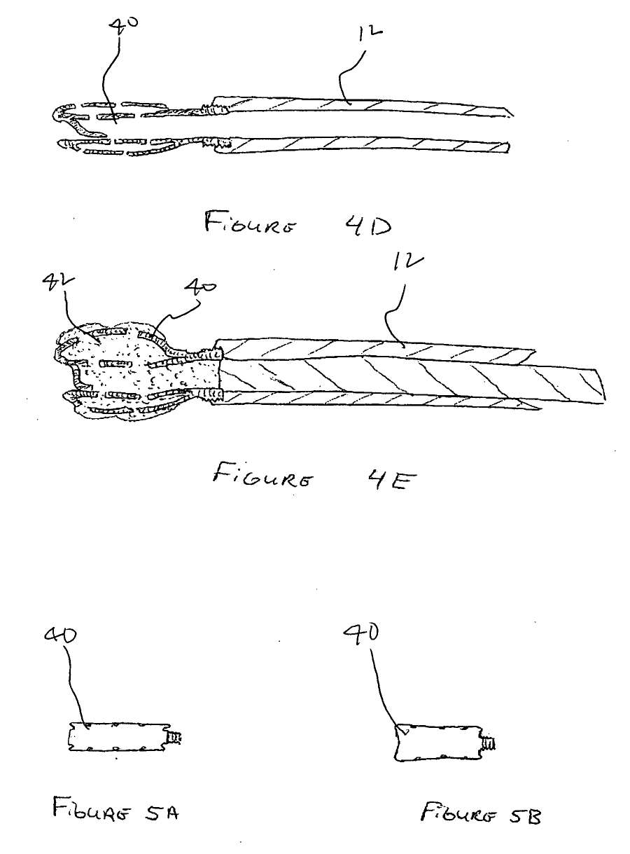

FIG. 2A is a drawing depicting the preferred embodiment of the present invention. Device 10 includes a flexible wire 20, a catheter 12, and a porous terminal component 22. Catheter 12 is attached to terminal component 22. For example, catheter 12 and terminal component 22 may be threaded together. Alternative mechanisms may be used to temporarily connect the two together, such as an adhesive. Heat could be used to disconnect the two. Heat sensitive biologic adhesive including fibrin glue (Tisscel) or BioDisc or BioGlue by Cyro-Life may be used to connect the catheter to the terminal component. The exothermic reaction of the curing PMMA could generate the heat required to release the catheter. Other temperature dependent shape memory fastening technology could be used, such as nitinol. In addition, a cutting device could be placed into the catheter to release the terminal component. This cutting device could include a right knife tip or a heated tip.

The terminal component may be made from a material that expands. The device could be made of bio-resorbable materials including polylactic acid (PLA), polyglycolic acid (PGA), poly (ortho esters), poly (glycolide-co-trimethylene carbonate), poly-L-lactide-co-6-caprolactone, polyanhydrides, poly-n-dioxanone, and poly (PHB-hydroxyvaleric acid). It may also be constructed to allow more expansion of the device in a cranial to caudal direction than in a radial direction. The device may also be made of elastic or inelastic materials. The device is preferably made of polymers.

FIG. 2B is an embodiment of the device in which the guide wire 20 runs up into the terminal component 22 at the tip of the catheter 12. In FIG. 2C, guide wire 20 has been removed from device 10 and has been replaced by a syringe 26 filled with PMMA. In FIG. 2D, PMMA 28 can be seen escaping from the holes in PMMA 28 is forced into terminal component 22 by activating the syringe 26. PMMA 28 is forced into component 22 faster than PMMA 28 can escape from the holes in component 22, causing component 22 to expand. PMMA 28 hardens before all of the material escapes from terminal component 22.

FIG. 3A shows the device 10 before it is inserted into a fractured vertebra 28. FIG. 3B shows device 10 in position in the vertebra. FIG. 3C shows device 10 where syringe 26 has been activated to cause PMMA to fill terminal component 22. Pressure should be maintained on the syringe until the PMMA has at least partially cured. Device 10 may be inserted through the pedicle of vertebra 28. Alternatively, device 10 may be placed into the lateral, posterior-lateral, or anterior portion of the vertebral body. Fluoroscopy or other navigational tools such as CT imaging may be used to aid in the placement of device 10.

FIG. 3C shows device 10 in use in a fractured vertebra. Guide wire 20 has been removed and a syringe 26 has been connected to catheter 12. PMMA 26 is seen extruding from the holes in terminal component 22, which has expanded the fractured vertebra. The holes in terminal component 22 are preferably located over the cranial and the caudal portions of component 22. Alternatively, the holes may be placed over the entire surface of component 22. In the preferred embodiment, holes are not placed over the posterior or dorsal portion of terminal component 22. PMMA that extends through the dorsal portion of the device could extend into the spinal canal, which could compress the spinal cord. Expansion of component 22 can be controlled by varying the number of holes in terminal component 22, the size of the holes in component 22, the rate at which the PMMA is forced into component 22. Also consider the rate at which PMMA cures, the viscosity of the PMMA, and perhaps the location of the holes in component 22. For example, component 22 could be expanded more with a partially cured PMMA, or quick curing PMMA that is injected into a component with holes that only occupy a small area of the component.

In FIG. 3D, catheter 12 has been disconnected from component 22. The PMMA that extends through component 22 into the vertebra prevents rotation of terminal component 22 as the catheter 12 is unscrewed from component 22. The catheter 12 is then removed from the patient's body. The PMMA and expanded terminal component 22 have restored the height of the fractured vertebra.

FIG. 4A shows the cross section of a vertebra 30, a needle 36 and a guide wire 34. FIG. 4B shows the vertebra 30, the guide wire 34 and a dilator 36. The guide wire 34 was inserted into the vertebra 30 through the needle 36 and the needle 36 removed. Dilator 36 is then passed over the guide wire 34 to enlarge the opening in vertebra 30. FIG. 4C shows a device 38 having a terminal component 40. Device 38 is passed over the guide wire 34. The hole in device 38 contains a one-way valve. The valve allows device 28 to be passed over guide wire 34. The valve also prevents PMMA from passing out of the tip of the device. PMMA is injected into the terminal component 40, and passes through the holes in component 40. FIG. 4E shows that PMMA 42 has been injected into the device 40. A plunger-like component has been passed into the catheter, and forces the PMMA 42 within the catheter into component 40. The plunger and catheter are then removed after the PMMA hardens.

An alternative embodiment of component 40 is shown in FIGS. 5A and 5B. Referring now to FIG. 5A, this terminal component requires less pressure to expand the device in a superior to inferior direction than to expand the device in a radial direction. Inelastic bands could be placed around the circumference of device 40. The superior and inferior positions of device 40 may use bellow-like components which allow for expansion of the device in a superior to inferior direction. The top and bottom of device 40 may be made of elastic material. FIG. 5B shows device 40 has been expanded in a superior to inferior direction.

What is claimed is:

1) A method for performing kyphoplasty, comprising the steps of:
inserting a catheter having a balloon at its distal end into a fractured vertebra;
injecting a substance capable of hardening through said catheter to inflate said balloon;
waiting for the substance to harden;
removing the catheter from the balloon and out of the patient.

2) The method of claim 1, wherein the substance is PMMA.

3) The method of claim 1, wherein the substance is hydroxyapatite.
